(19) United States
(12) Patent Application Publication
(10) Pub. No.: US 2023/0389904 A1
(43) Pub. Date: Dec. 7, 2023

(54) Male External Elimination Module for Urinary Collection

(71) Applicant: Debra Ware, Orange, TX (US)

(72) Inventor: Debra Ware, Orange, TX (US)

(21) Appl. No.: 18/206,573

(22) Filed: Jun. 6, 2023

Related U.S. Application Data
(60) Provisional application No. 63/349,309, filed on Jun. 6, 2022.

(51) Int. Cl. A61B 10/00; A61F 5/453; A61F 5/443; A61F 5/44

(52) U.S. Cl. CPC A61B 10/007; A61F 5/453; A61F 5/443; A61F 5/4408

(57) ABSTRACT

A replacement for the external male condom catheter, including a circular or oval apparatus that encloses the penis, a sample port for urine sample collection, a urine collection chamber, and a drainage tube.

This application relates back to provisional application U.S. 63/349,309 filed on Jun. 6, 2023, and incorporates the application in its entirety.

### SUMMARY

A male fluid collection device, to remove fluid from the male urinary system without a Foley catheter or a traditional condom catheter. The MEEMUC has a collection chamber with a sample port for collection of a clean urine specimen.

### BACKGROUND

Hospitalized men are subject to infections due to indwelling Foley catheters. As an alternative, to reduce this problem, physicians often write orders for condom catheters which use the same gravity drainage bags as Foley catheters. However, condom catheters do not stay in place, falling off when the patient moves or is moved. Typically, the male condom catheter is attached to a standard Foley catheter gravity drainage bag, which frequently does not work very well. There is often leakage, and because the male condom catheters fall off, they must also be replaced. Because of the leakage, an accurate measure of urine cannot be obtained, and they fall off so easily that replacing the male condom catheter may be required as often as 6 times in a single shift. In order to get a clean urine sample, the male condom catheter must be removed. The frequent replacement increases costs for both patients and hospitals. Additionally, wet bedding is unpleasant and requires laundering, and failure to notice and appropriately replace wet bedding can cause bed sores for the patient.

### DETAILED DESCRIPTION

In the following description, numerous specific details are set forth to clearly describe the embodiments disclosed herein. However, one skilled in the art will understand that some well known features have not been described in detail so as not to obscure the invention.

### BRIEF DESCRIPTION OF DRAWINGS

FIG. 1 shows a side perspective view of the urine collection device hose.

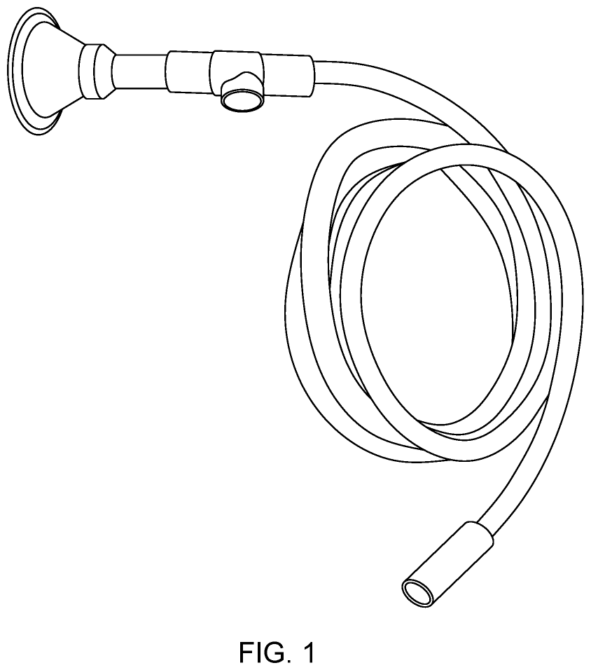

FIG. 2 shows a side perspective view of the urine collection device hose connected to the container.

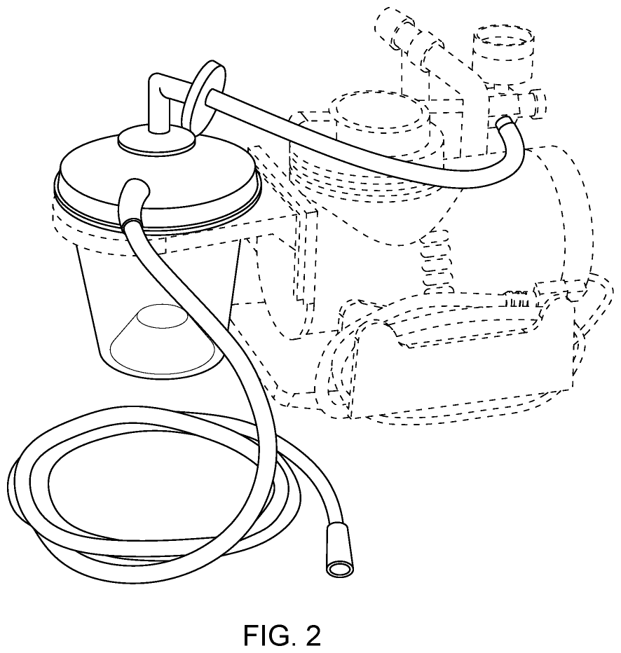

In the following description, like reference numbers are used to identify like elements. Furthermore, the drawings are intended to illustrate major features of the exemplary embodiments in a diagrammatic manner. The drawings are not intended to depict every feature of every implementation and are not drawn to scale.

The invention comprises 4 essential parts: a circular or oval shaped apparatus intended to enclose the penis (1), a sampling port for urine collection (2), a urine collection chamber (3), and drainage tubing (4). Not part of the invention, and therefore not shown, is the portable vacuum source or gravity drain bag the tubing connects to. The circular apparatus (1) may come in various sizes, such as small, medium, and large, in order to have appropriate dimensions for different patients. The tubing ranges from 2-3 centimeters in outer diameter, and is approximately 6 feet long, so as to be sufficient to the task without having so much length that it becomes hard to work around. The sampling port is sized appropriately for a 10 cc syringe to be used to collect a urine specimen.

The MEEMUC may be connected to the patient via adhesive, as is known for condom catheters. In the preferred embodiment, the MEEMUC is not connected to the patient via glue, instead being retained in position by low grade vacuum. This not only avoids problems from the glue providing insufficient force to retain position relative to the patient, but also avoids potential chemical irritation or reaction from the glue.

While illustrative embodiments of the invention have been shown and described, variations and alternative embodiments will occur to those skilled in the art. Such variations and alternative embodiments can be made without departing from the scope of the invention as defined in the claims.

As used in this specification and the appended claims, the singular forms “a” and “an” indicate a single element, while “the” may refer back to single or plural referents. Unless defined otherwise, all technical and scientific terms used herein have the same meaning as commonly understood by one of ordinary skill in the art to which the disclosure pertains.

The above detailed description of exemplary and preferred embodiments is presented for the purposes of illustration and disclosure in accordance with the requirements of the law. It is intended to be exemplary but not exhaustive, and is not intended to limit the invention to the precise forms described, but only to enable others skilled in the art to understand how the invention may be suited for a particular use of implementation. No limitation is intended by the description of exemplary embodiments which may have included tolerances, feature dimensions, specific operating conditions, engineering specifications, or the like, and which may vary between implementations or with changes to the state of the art, and no such limitation should be implied therefrom. Applicant has made this disclosure with respect to the current state of the art, but also contemplates advancements and that adaptations in the future may take into consideration those advancements in accordance with the then current state of the art. It is intended that the scope of the invention be defined by the Claims as written and equivalents as applicable. Reference to a claim element in the singular is not intended to mean “one and only one” unless explicitly so stated. No claim element herein is intended to be construed under the provisions of 35 U.S.C. 112(f), unless the element is expressly recited using the exact phrase “means for . . . ” and no method or process step herein is to be construed under the provisions of 35 U.S.C. section 112(f) unless the step, or steps, are expressly recited using the exact phrase “step(s) for . . . ”.

1. A male fluid collection device for removing urine from the body, comprising:
a circular or oval shaped apparatus that encloses the penis of a patient when in use,
a sampling port for collection of a clean urine specimen;
a urine collection chamber, for collection of a clean urine specimen; and
tubing, which is connected to the apparatus that encloses the penis when in use.

2. The male fluid collection device for removing urine from the body of claim 1, wherein:
the urine collection chamber is directly attached to apparatus that encloses the penis when in use at one end, and directly attached to the tubing at the opposite end of the urine collection chamber from the attachment to the apparatus that encloses the penis.

3. The male fluid collection device for removing urine from the body of claim 1, wherein:
the sampling port is located in the urine collection chamber and is configured to have a 10 cc syringe used to collect the clean urine specimen.

4. The male fluid collection device for removing urine from the body of claim 1, wherein the end of the tubing that is not attached to the collection chamber is configured to attach to at least one of a portable vacuum source and a gravity drain bag.

5. The male fluid collection device for removing urine from the body of claim 1, wherein the apparatus that encloses the penis when in use is configured to attach to the patient via adhesive or vacuum pressure.
